(19) United States
(12) Patent Application Publication
(10) Pub. No.: US 2009/0054490 A1
(43) Pub. Date: Feb. 26, 2009

(54) METHODS FOR THE TREATMENT OF GERD WITH MGLUR5 ANTAGONISTS

(76) Inventors: Georg Jaeschke, Basel (CH); Sabine Kolczewski, Loerrach (DE); Will Spooren, Franken (FR); Eric Vieira, Frenkendorf (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/190,614

(22) Filed: Aug. 13, 2008

(30) Foreign Application Priority Data
Aug. 20, 2007 (EP) 07114582.5

(51) Int. Cl. A61K 31/444; A61K 31/4439; A61P 1/04

(52) U.S. Cl. 514/333; 514/341

(57) ABSTRACT

The present invention relates to methods for the treatment, prevention and/or delay of progression of gastro-esophageal reflux disease (GERD) by administering compounds that act as antagonists of metabotropic glutamate type-5 receptors (mGluR5 receptor antagonists), for example compounds of formula (I)

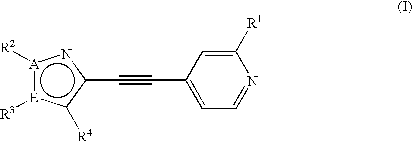

wherein A, E, R¹, R², R² and R⁴ are as defined in the specification.

### PRIORITY TO RELATED APPLICATION(S)

This application claims the benefit of European Patent Application No. 07114582.5, filed Aug. 20, 2007, which is hereby incorporated by reference in its entirety.

### BACKGROUND OF THE INVENTION

The metabotropic glutamate receptors (mGluR) are G-protein coupled receptors that are involved in the regulation and activity of many synapses in the central nervous system (CNS). Eight metabotropic glutamate receptor subtypes have been identified and are subdivided into three groups based on sequence similarity. Group I consists of mGluR1 and mGluR5. These receptors activate phospholipase C and increase neuronal excitability.

Group II, consisting of mGluR2 and mGluR3 as well as group III, consisting of mGluR4, mGluR6, mGluR7 and mGluR8 are capable of inhibiting adenylyl cyclase activity and reduce synaptic transmission. Several of the receptors also exist in various isoforms, occurring by alternative splicing (Chen, C-Y et al., Journal of Physiology (2002),538. 3, pp.773-786,-Pin, J-P et al., European Journal of Pharmacology (1999), 375, pp.277-294; Brauner-Osborne, H et al. Journal of Medicinal Chemistry (2000), 43, pp. 2609-2645; Schoepp, D. D., lane D. E. Monn J. A. Neuropharmacology (1999), 38, pp.1431-1476).

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer., 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

According to Blackshaw L. A. et al., presentation at the conference Neurogastroenterology & Motility, Madison, Wis., 14 Nov. 2001, metabotropic glutamate receptors of group II and group III, i.e. mGluR2, mGluR3, mGluR4, mGluR6, mGluR7 and mGluR8 may be involved in selective inhibitory modulation of peripheral mechanosensory endings.

WO 03/047581 discloses mGluR5 antagonists and their use as pharmaceuticals, especially in the treatment of nervous system disorders. WO 05/044265, WO 05/044266, WO 05/044267 and WO 07/006530 disclose mGluR5 antagonists and their use as pharmaceuticals in the treatment or prevention of gastro-esophageal reflux disease (GERD).

The known medication for treatment of gastro-esophageal reflux disease (GERD) has some drawbacks in terms of limited efficacy, tolerability, convenience, and safety.

### SUMMARY OF THE INVENTION

The present invention provides methods for the treatment, prevention and/or delay of progression of gastro-esophageal reflux disease (GERD) by administering compounds that act as antagonists of metabotropic glutamate type-5 receptors (mGluR5 receptor antagonists)

Compounds of formula (I)

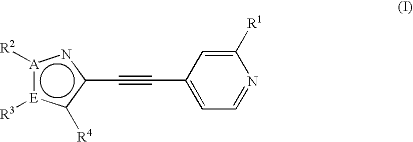

wherein
one of A or E is N and the other is C;
R¹ is halogen;
R² is C₁-C₆-alkyl;
R³ is phenyl, pyridinyl, pyrazinyl, pyrimidinyl or pyridazinyl, each of which is optionally substituted by one, two or three substituents, selected from the group consisting of halogen, C₁-C₆-alkyl, C₁-C₆-alkoxy, C₁-C₆-haloalkyl and C1-C₆-haloalkoxy; and
   R⁴ is CHF₂, CF₃, CH₂OH or C₁-C₆-alkyl; in free base or acid addition salt form are highly effective in the treatment, prevention and/or delay of progression of gastro-esophageal reflux disorder (GERD).

### DETAILED DESCRIPTION OF THE INVENTION

Preferred compounds of formula I are those compounds of formulae Ia and Ib:

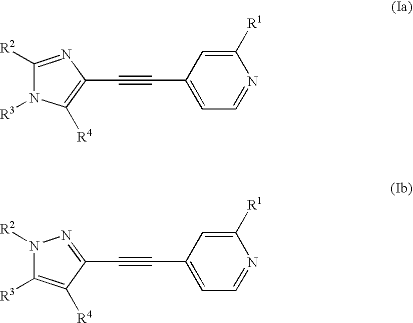

wherein R¹, R², R³ and R⁴ are as defined herein above.

In the compounds of formulae I, Ia or Ib, according to the invention, R¹ is preferably halogen; R² is preferably methyl or i-propyl; R³ is preferably selected from phenyl or pyridinyl which are optionally substituted by one or more chloro, fluoro, C₁-C₆-alkyl, C₁-C₆-alkoxy, C₁-C₆-haloalkyl or C₁-C₆-haloalkoxy; and R⁴ is preferably methyl, CHF₂ or CH₂OH.

Preferred are those compounds of formula Ia, wherein, R³ is unsubstituted or substituted pyridinyl, wherein the substituents are selected from chloro, fluoro, CF₃ and C₁-C₆-alkyl, for example the following compounds:
2-[4-(2-Chloro-pyridin-4-ylethynyl)-2,5-dimethyl-1H-imidazol-1-yl]-5-methyl-pyridine;
2-Chloro-5-[4-(2-Chloro-pyridin-4-ylethynyl)-2,5-dimethyl-1H-imidazol-1-yl]-pyridine;
2-[4-(2-Chloro-pyridin-4-ylethynyl)-2,5-dimethyl-1H-imidazol-1-yl]-6-methyl-4-trifluoromethyl-pyridine;
2-[4-(2-Chloro-pyridin-4-ylethynyl)-2,5-dimethyl-1H-imidazol-1-yl]-6-methyl-pyridine;
2-[4-(2-Chloro-pyridin-4-ylethynyl)-2,5-dimethyl-1H-imidazol-1-yl]-6-(trifluoromethyl)-pyridine; and
3-[4-(2-Chloro-pyridin-4-ylethynyl)-2,5-dimethyl-1H-imidazol-1-yl]-5-fluoro-pyridine.

Especially preferred are further those compounds of formula Ia, wherein, R³ is phenyl, substituted by one or more chloro, fluoro, CF₃, C₁-C₆-alkyl, C₁-C₆-alkoxy, C₁-C₆-haloalkyl or C₁-C₆-haloalkoxy, for example the following compounds:
2-Chloro-4-[1-(4-fluoro-phenyl)-2,5-dimethyl-1H-imidazol-4-ylethynyl]-pyridine;
2-Chloro-4-[1-(2,4-difluoro-phenyl)-2,5-dimethyl-1H-imidazol-4-ylethynyl]-pyridine;
2-Chloro-4-[1-(3,5-difluoro-phenyl)-2,5-dimethyl-1H-imidazol-4-ylethynyl]-pyridine;
2-Chloro-4-[1-(4-fluoro-2-methyl-phenyl)-2,5-dimethyl-1H-imidazol-4-ylethynyl]-pyridine;
   2-Chloro-4-[1-(4-fluoro-3-methyl-phenyl)-2,5-dimethyl-1H-imidazol-4-ylethynyl]-pyridine;
2-Chloro-4-(2,5-dimethyl-1-p-tolyl-1H-imidazol-4-ylethynyl)-pyridine;
2-Chloro-4-[1-(3-chloro-4-methyl-phenyl)-2,5-dimethyl-1H-imidazol-4-ylethynyl]-pyridine;
2-Chloro-4-[1-(3-fluoro-4-methoxy-phenyl)-2,5-dimethyl-1H-imidazol-4-ylethynyl]-pyridine;
2-Chloro-4-[1-(4-methoxy-phenyl)-2,5-dimethyl-1H-imidazol-4-ylethynyl]-pyridine;
2-Chloro-4-[2,5-dimethyl-1-(4-trifluoromethoxy-phenyl)-1H-imidazol-4-ylethynyl]-pyridine;
2-Chloro-4-[2,5-dimethyl-1-(3-trifluoromethoxy-phenyl)-1H-imidazol-4-ylethynyl]-pyridine;
2-Chloro-4-[2,5-dimethyl-1-(4-trifluoromethyl-phenyl)-1H-imidazol-4-ylethynyl]-pyridine;
2-Chloro-4-[2,5-dimethyl-1-(3-methyl-4-trifluoromethoxy-phenyl)-1H-imidazol-4-ylethynyl]-pyridine;
2-Chloro-4-[1-(4-chloro-phenyl)-2,5-dimethyl-1H-imidazol-4-ylethynyl]-pyridine;
2-Chloro-4-[1-(3-chloro-2-fluoro-phenyl)-2,5-dimethyl-1H-imidazol-4-ylethynyl]-pyridine;
2-Chloro-4-[2,5-dimethyl-1-(3-trifluoromethyl-phenyl)-1H-imidazol-4-ylethynyl]-pyridine;
2-Chloro-4-[1-(3-chloro-4-fluoro-phenyl)-2,5-dimethyl-1H-imidazol-4-ylethynyl]-pyridine;
2-Chloro-4-[2,5-dimethyl-1-(2-methyl-4-trifluoromethoxy-phenyl)-1H-imidazol-4-ylethynyl]-pyridine;
2-Chloro-4-[5-difluoromethyl-1-(4-fluoro-phenyl)-2-methyl-1H-imidazol-4-ylethynyl]-pyridine;
[5-(2-Chloro-pyridin-4-ylethynyl)-3-(4-fluoro-phenyl)-2-methyl-3H-imidazol-4-yl]-methanol;
2-Chloro-4-[1-(4-methoxy-3-trifluoromethyl-phenyl)-2,5-dimethyl-1H-imidazol-4-ylethynyl]-pyridine;
2-Chloro-4-[1-(3,5-difluoro-4-methoxy-phenyl)-2,5-dimethyl-1H-imidazol-4-ylethynyl]-pyridine;
2-Chloro-4-[1-(4-methoxy-3-trifluoromethoxy-phenyl)-2,5-dimethyl-1H-imidazol-4-ylethynyl]-pyridine;
2-Chloro-4-[1-(3-methoxy-4-trifluoromethoxy-phenyl)-2,5-dimethyl-1H-imidazol-4-ylethynyl]-pyridine;
2-Chloro-4-[1-(4-fluoro-2-trifluoromethoxy-phenyl)-2,5-dimethyl-1H-imidazol-4-ylethynyl]-pyridine;
2-Chloro-4-[1-(2-fluoro-4-trifluoromethoxy-phenyl)-2,5-dimethyl-1H-imidazol-4-ylethynyl]-pyridine;
2-Chloro-4-[2,5-dimethyl-1-(4-methyl-3-trifluoromethyl-phenyl)-1H-imidazol-4-ylethynyl]-pyridine;
2-Chloro-4-[2,5-dimethyl-1-(3-methyl-4-trifluoromethyl-phenyl)-1H-imidazol-4-ylethynyl]-pyridine;
2-Chloro-4-[2,5-dimethyl-1-(3-methyl-5-trifluoromethyl-phenyl)-1H-imidazol-4-ylethynyl]-pyridine;
2-Chloro-4-[1-(3-methoxy-5-trifluoromethyl-phenyl)-2,5-dimethyl-1H-imidazol-4-ylethynyl]-pyridine;
2-Chloro-4-[1-(3-methoxy-4-trifluoromethyl-phenyl)-2,5-dimethyl-1H-imidazol-4-ylethynyl]-pyridine;
2-Chloro-4-[1-(3,5-dichloro-phenyl)-2,5-dimethyl-1H-imidazol-4-ylethynyl]-pyridine;
2-Chloro-4-[1-(3-chloro-5-methyl-phenyl)-2,5-dimethyl-1H-imidazol-4-ylethynyl]-pyridine;
2-Chloro-4-[1-(3-fluoro-5-methyl-phenyl)-2,5-dimethyl-1H-imidazol-4-ylethynyl]-pyridine;
2-Chloro-4-[1-(3-chloro-5-methoxy-phenyl)-2,5-dimethyl-1H-imidazol-4-ylethynyl]-pyridine; and
2-Chloro-4-[1-(3-fluoro-5-methoxy-phenyl)-2,5-dimethyl-1H-imidazol-4-ylethynyl]-pyridine.

Also preferred are further those compounds of formula Ib, wherein R³ is phenyl, substituted by one or more fluoro; especially the following compound: 2-Chloro-4-[5-(4-fluoro-phenyl)-1,4-dimethyl-1H-pyrazol-3-ylethynyl]-pyridine.

The compounds of formula Ia of the invention may be prepared according to the procedures described in WO 2005/118568.

In addition to the mGluR5 receptor antagonists described hereinbefore, further suitable mGluR5 receptor antagonists can be selected from the compounds and groups of compounds as specifically disclosed in WO 2004/108701, WO 2004/080998, WO 2005/118568, WO 2004/111040, WO 2005/003117, WO 2005/023795, WO 2006/074884, WO 2006/094639, WO 2006/094691, WO 2007/039512, WO 2007/054436, PCT/EP2007/051165, WO 99/08678, WO 00/58285, WO 00/63166, EP 1074549 A1, WO 01/27070, WO 02/02554, WO 02/06288, WO 02/06254, WO 02/46166, WO 02/051418, WO 02/092086, WO 02/094795, WO 02/098864, and WO 2004/056814 and especially in the claims thereof.

It will be understood that in the discussion hereinafter, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredient or a pharmaceutically acceptable salt thereof may also be used in form of a hydrate or include other solvents used for crystallization. The compounds of formula (I) and their manufacture are already known, e.g. from WO 04/108701 and WO 2005/118568.

The following definitions of general terms used in the present description apply irrespective of whether the terms in question appear alone or in combination.

For the purpose of this invention, the term “antagonist” should be understood as including full antagonists, inverse agonists, non-competitive antagonists or competitive antagonists, as well as partial antagonists, whereby a “partial antagonist” should be understood as a compound capable of partially, but not fully, in-activating the metabotropic glutamate receptor 5.

The wording “TLESR”, transient lower esophageal sphincter relaxations, is herein defined in accordance with Mittal, R. K., Holloway, R. H., Penagini, R., Blackshaw, L. A., Dent, J., 1995; Transient lower esophageal sphincter relaxation. Gastroenterology 109, pp. 601-610.

The wording “reflux” is defined as fluid from the stomach being able to pass into the esophagus, since the mechanical barrier is temporarily lost at such times.

The wording “GERD”, gastro-esophageal reflux disease, is defined in accordance with van Heerwarden, M. A., Smout A. J. P. M., 2000; Diagnosis of reflux disease. Bailliere's Clin. Gastroenterol. 14, pp. 759-774.

The term “C₁-C₆-alkyl” used in the present description denotes straight-chain or branched saturated hydrocarbon residues with 1 to 6 carbon atoms, preferably with 1 to 4 carbon atoms, such as methyl, ethyl, n-propyl, i-propyl, n-butyl, t-butyl and the like.

The term “C₁-C₆-alkoxy” denotes a lower alkyl residue in the sense of the foregoing definition bound via an oxygen atom. Examples of “C₁-C₆-alkoxy” residues include methoxy, ethoxy, isopropoxy and the like.

The term “halogen” denotes fluorine, chlorine, bromine and iodine.

The term “C₁-C₆-haloalkyl” denotes a lower alkyl group as defined above which is substituted by one or more halogen atom. Examples of lower haloalkyl include but are not limited to methyl, ethyl, propyl, isopropyl, isobutyl, sec-butyl, tert-butyl, pentyl or n-hexyl substituted by one or more Cl, F, Br or I atom(s) as well as those groups specifically illustrated by the examples herein below. Preferred lower haloalkyl are difluoro- or trifluoro-methyl or ethyl.

The term “C₁-C₆-haloalkoxy” denotes lower alkoxy group as defined above which is substituted by one or more halogen atom. Examples of C₁-C₆-haloalkoxy include but are not limited to methoxy or ethoxy, substituted by one or more Cl, F, Br or I atom(s) as well as those groups specifically illustrated by the examples herein below. Preferred lower haloalkoxy are difluoro- or trifluoro-methoxy or ethoxy.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable salt” refers to any salt derived from an inorganic or organic acid or base. Such salts include: acid addition salts formed with inorganic acids such as hydrochloric acid, hydrobromic acid, sulfuric acid, nitric acid, phosphoric acid; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid or trimethylacetic acid.

“Therapeutically effective amount” means an amount that is effective to prevent, alleviate or ameliorate symptoms of disease or prolong the survival of the subject being treated.

In accordance with the present invention, compounds of formula (I) are useful in the treatment, prevention and/or delay of progression of gastro-esophageal reflux disease (GERD).

Gastro-Esophaqeal Reflux Disease (GERD) results from the retrograde flow of gastric contents into the esophagus. It is the most common ailment in the upper gastro-intestinal tract; its cardinal feature and symptom is commonly known as “heartburn”. A major factor considered for GERD is an incompetence of the Lower Esophageal Sphincter that opens transiently and allows passage of material (e.g. meal, acidic fluid or bile), from the stomach into the esophagus. This motor event denominated Transient Lower Esophageal Sphincter Relaxation (TLESR) occurs more often in patients suffering from GERD than in healthy subjects and occurs more often in infants with regurgitation. Current standard therapies in GERD aim at suppressing gastric acid secretion or enhancing gastrointestinal motility to limit the exposure of the esophagus to acidic gastric contents. Frequent exposure of the esophageal mucosa to acid can trigger pain (often perceived as heartburn) and lead to erosions. It can also lead to extra-esophageal disorders such as asthma, cough and laryngitis. To date, there is no treatment available which reduces the occurrence of TLESRs and, thereby, the symptoms associated with GERD or regurgitation in infants.

A further aspect of the invention is pharmaceutical compositions containing a therapeutically effective amount of a compound of formula (I) and a pharmaceutically acceptable carrier.

In a further aspect, the invention provides a method for the treatment, prevention and/or delay of progression of gastro-esophageal reflux disease (GERD) in a subject in need of such treatment, which comprises administering to said subject a therapeutically effective amount of a compound of formula (I) described hereinbefore in free base or pharmaceutically acceptable salt form.

In a further aspect, the invention provides a pharmaceutical composition comprising a compound of formula (I) described hereinbefore in free base or pharmaceutically acceptable salt form for the treatment, prevention and/or delay of progression of gastro-esophageal reflux disease (GERD).

The present invention also provides pharmaceutical compositions containing compounds of the invention, or pharmaceutically acceptable salts thereof, and a pharmaceutically acceptable carrier. Such pharmaceutical compositions can be in the form of tablets, coated tablets, dragees, hard and soft gelatin capsules, solutions, emulsions or suspensions. The pharmaceutical compositions can also be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acid or its salts and the like can be used, for example, as such as carriers for tablets, coated tablets, dragees and hard gelatin capsules. Suitable carriers for soft gelatin capsules are, for example, vegetable oils, waxes, fats, semi-solid and liquid polyols and the like; depending on the nature of the active substance no carriers are, however, usually required in the case of soft gelatin capsules. Suitable carriers for the production of solutions and syrups are, for example, water, polyols, sucrose, invert sugar, glucose and the like. Adjuvants, such as alcohols, polyols, glycerol, vegetable oils and the like, can be used for aqueous injection solutions of water-soluble salts of compounds of formula I, but as a rule are not necessary. Suitable carriers for suppositories are, for example, natural or hardened oils, waxes, fats, semi-liquid or liquid polyols and the like.

In addition, the pharmaceutical compositions can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

In another aspect, the invention provides a pharmaceutical composition comprising both a compound of formula (I) described hereinbefore in free base or pharmaceutically acceptable salt form and at least one anti-secretory agent. The anti-secretory agent is preferably selected from the group consisting of proton pump inhibitors (PPI) and histamine H2 receptor antagonists.

Proton pump inhibitors act by irreversibly blocking the hydrogen/potassium adenosine triphosphatase enzyme system (gastric proton pump) of the gastric parietal cell. The proton pump is the terminal stage in gastric acid secretion, being directly responsible for secreting H+ ions into the gastric lumen. The proton pump inhibitors are given in an inactive form. The inactive form is neutrally charged (lipophilic) and readily crosses cell membranes into intracellular compartments (like the parietal cell canaliculus) that have acidic environments. In an acid environment, the inactive drug is protonated and rearranges into its active form. As described above, the active form will covalently and irreversibly bind to the gastric proton pump, deactivating it. Clinically useful examples of proton pump inhibitors include but are not limited to: Omeprazole (brand names: Losec®, Prilosec®, Zegerid®), Lansoprazole (brand names: Prevacid®, Zoton®, Inhibitol®), Esomeprazole (brand names: Nexium®), Pantoprazole (brand names: Protonix®, Somac®, Pantoloc®) and Rabeprazole as well as mixtures thereof.

The H2 receptor antagonists are competitive inhibitors of histamine at the parietal cell H2 receptor. They suppress the normal secretion of acid by parietal cells and the meal-stimulated secretion of acid. They accomplish this by two mechanisms: histamine released by ECL cells in the stomach is blocked from binding on parietal cell H2 receptors which stimulate acid secretion, and other substances that promote acid secretion (such as gastrin and acetylcholine) have a reduced effect on parietal cells when the H2 receptors are blocked. Suitable examples of histamine H2 receptor antagonists inhibitors include but are not limited to cimetidine, ranitidine, famotidine, burimamide, metiamide, nizatidine, tiotidine and oxmetidine as well as mixtures thereof.

The pharmaceutical compositions of the present invention typically comprise, by weight, from about 0.1% to about 99.8% of the anti-secretory agent, preferably from about 0.1% to about 75%, and most preferably from about 1% to about 50%.

When anti-secretory agents are used, for instance those described hereinbefore, the gastroduodenal pH will be elevated to around pH 4. It is known, that an elevated gastroduodenal pH can reduce the bioavailability of basic compounds. Surprisingly, there is no significant impact on the bioavailability of the mGluR5 receptor antagonists described hereinbefore as useful for the treatment, prevention and/or delay of progression of gastro-esophageal reflux disease (GERD) when clinical dosages of the anti-secretory agents are concurrently administered.

As mentioned earlier, compositions containing a compound of formula I or pharmaceutically acceptable salts thereof and a therapeutically inert excipient are also an object of the present invention, as is a process for the production of such medicaments which comprises bringing one or more compounds of formula I or pharmaceutically acceptable salts thereof and, if desired, one or more other therapeutically valuable substances into a galenical dosage form together with one or more therapeutically inert carriers.

The following examples are provided to further elucidate the invention.

The pharmacological activity of the compounds of formula (I) described hereinbefore was tested using the following method:

For binding experiments, cDNA encoding human mGlu5a receptor was transiently transfected into EBNA cells using a procedure described by Schlaeger and Christensen [Cytotechnology 15:1-13 (1998)]. Cell membrane homogenates were stored at −80° C. until the day of assay where upon they were thawed and resuspended and polytronised in 15 mM Tris-HCl, 120 mM NaCl, 100 mM KCl, 25 mM CaCl₂, 25 mM MgCl₂ binding buffer at pH 7.4 to a final assay concentration of 20 g protein/well.

Saturation isotherms were determined by addition of twelve [³H]MPEP concentrations (0.04-100 nM) to these membranes (in a total volume of 200 μl) for 1 h at 4° C. Competition experiments were performed with a fixed concentration of [³H]MPEP (2 nM) and IC₅₀ values of test compounds evaluated using 11 concentrations (0.3-10,000 nM). Incubations were performed for 1 h at 4° C.

At the end of the incubation, membranes were filtered onto unifilter (96-well white microplate with bonded GF/C filter preincubated 1 h in 0.1% PEI in wash buffer, Packard BioScience, Meriden, Conn.) with a Filtermate 96 harvester (Packard BioScience) and washed 3 times with cold 50 mM Tris-HCl, pH 7.4 buffer. Nonspecific binding was measured in the presence of 10 M MPEP. The radioactivity on the filter was counted (3 min) on a Packard Top-count microplate scintillation counter with quenching correction after addition of 45 μl of microscint 40 (Canberra Packard S.A., Zutrich, Switzerland) and shaking for 20 min.

For functional assays, [Ca²⁺]i measurements were performed as described previously by Porter et al. [Br. J. Pharmacol. 128:13-20 (1999)] on recombinant human mGlu5a receptors in HEK-293 cells. The cells were dye loaded using Fluo 4-AM (obtainable by FLUKA, 0.2 μM final concentration). [Ca²⁺]i measurements were performed using a fluorometric imaging plate reader (FLIPR, Molecular Devices Corporation, La Jolla, Calif., USA). Antagonist evaluation was performed following a 5 min preincubation with the test compounds followed by the addition of a submaximal addition of agonist.

The inhibition (antagonists) curves were fitted with a four parameter logistic equation giving IC₅₀, and Hill coefficient using an iterative non linear curve fitting software (Xcel fit).

For binding experiments the Kᵢ values of the compounds tested are given. The Kᵢ value is defined by the following formula:

Kᵢ=IC₅₀/[1+L/K_{d}]

in which the IC₅₀ values are those concentrations of the compounds tested which cause 50% inhibition of the competing radioligand ([³H]MPEP). L is the concentration of radioligand used in the binding experiment and the K_{d} value of the radioligand is empirically determined for each batch of membranes prepared.

The compounds described hereinbefore are mGluR5a receptor antagonists. The activities of compounds of formula (I) as measured in the assay described above are in the range of Kᵢ<75 nM.

| | | Kᵢ |
|---|---|---|
| | | (mGluR5) |
| No. | Name | nM |
| 1 | 2-Chloro-4-[1-(4-fluoro-phenyl)-2,5-dimethyl-1H-imidazol-4- | 29 |
| | ylethynyl]-pyridine | |
| 2 | 2-Chloro-4-[1-(2,4-difluoro-phenyl)-2,5-dimethyl-1H-imidazol-4- | 22 |
| | ylethynyl]-pyridine | |
| 4 | 2-Chloro-4-[1-(3,5-difluoro-4-methyl-phenyl)-2-methyl-1H-imidazol- | 6 |
| | 4-ylethynyl]-pyridine | |
| 5 | 2-Chloro-4-[1-(4-fluoro-2-methyl-phenyl)-2,5-dimethyl-1H-imidazol- | 50 |
| | 4-ylethynyl]-pyridine | |
| 6 | 2-Chloro-4-[1-(4-fluoro-3-methyl-phenyl)-2,5-dimethyl-1H-imidazol- | 35 |
| | 4-ylethynyl]-pyridine | |
| 7 | 2-Chloro-4-(2,5-dimethyl-1-p-tolyl-1H-imidazol-4-ylethynyl)-pyridine | 21 |
| 8 | 2-Chloro-4-[1-(3-chloro-4-methyl-phenyl)-2,5-dimethyl-1H-imidazol- | 10 |
| | 4-ylethynyl]-pyridine | |
| 9 | 2-Chloro-4-[1-(3-fluoro-4-methoxy-phenyl)-2,5-dimethyl-1H-imidazol- | 48 |
| | 4-ylethynyl]-pyridine | |
| 10 | 2-Chloro-4-[1-(4-methoxy-phenyl)-2,5-dimethyl-1H-imidazol-4- | 30 |
| | ylethynyl]-pyridine | |
| 11 | 2-Chloro-4-[2,5-dimethyl-1-(4-trifluoromethoxy-phenyl)-1H-imidazol- | 8 |
| | 4-ylethynyl]-pyridine | |
| 12 | 2-Chloro-4-[2,5-dimethyl-1-(3-trifluoromethoxy-phenyl)-1H-imidazol- | 14 |
| | 4-ylethynyl]-pyridine | |
| 13 | 2-Chloro-4-[2,5-dimethyl-1-(4-trifluoromethyl-phenyl)-1H-imidazol-4- | 44 |
| | ylethynyl]-pyridine | |
| 14 | 2-Chloro-4-[2,5-dimethyl-1-(3-methyl-4-trifluoromethoxy-phenyl)-1H- | 5 |
| | imidazol-4-ylethynyl]-pyridine | |
| 15 | 2-[4-(2-Chloro-pyridin-4-ylethynyl)-2,5-dimethyl-1H-imidazol-1-yl]-5- | 70 |
| | methyl-pyridine | |
| 16 | 2-Chloro-5-[4-(2-Chloro-pyridin-4-ylethynyl)-2,5-dimethyl-1H- | 86 |
| | imidazol-1-yl]-pyridine | |
| 17 | 2-[4-(2-Chloro-pyridin-4-ylethynyl)-2,5-dimethyl-imidazol-1-yl]-6- | 79 |
| | methyl-4-trifluoromethyl-pyridine | |
| 18 | 2-[4-(2-Chloro-pyridin-4-ylethynyl)-2,5-dimethyl-imidazol-1-yl]- | 108 |
| | pyrazine | |
| 19 | 2-Chloro-4-[1-(4-chloro-phenyl)-2,5-dimethyl-1H-imidazol-4- | 20 |
| | ylethynyl]-pyridine | |
| 20 | 2-Chloro-4-[1-(3-chloro-2-fluoro-phenyl)-2,5-dimethyl-1H-imidazol-4- | 28 |
| | ylethynyl]-pyridine | |
| 21 | 2-Chloro-4-[2,5-dimethyl-1-(3-trifluoromethyl-phenyl)-1H-imidazol-4- | 55 |
| | ylethynyl]-pyridine | |
| 22 | 2-Chloro-4-[1-(3-chloro-4-fluoro-phenyl)-2,5-dimethyl-1H-imidazol-4- | 31 |
| | ylethynyl]-pyridine | |
| 23 | 2-[4-(2-Chloro-pyridin-4-ylethynyl)-2,5-dimethyl-1H-imidazol-1-yl]-6- | 93 |
| | methyl-pyridine | |
| 24 | 2-[4-(2-Chloro-pyridin-4-ylethynyl)-2,5-dimethyl-1H-imidazol-1-yl]-6- | 26 |
| | (trifluoromethyl)-pyridine | |
| 26 | 2-Chloro-4-[5-(4-fluoro-phenyl)-1,4-dimethyl-1H-pyrazol-3-ylethynyl]- | 5.9 |
| | pyridine | |
| 27 | 2-Chloro-4-[5-difluoromethyl-1-(4-fluoro-phenyl)-2-methyl-1H- | 42.2 |
| | imidazol-4-ylethynyl]-pyridine | |
| 28 | [5-(2-Chloro-pyridin-4-ylethynyl)-3-(4-fluoro-phenyl)-2-methyl-3H- | 211 |
| | imidazol-4-yl]-methanol | |

The effectiveness of the compounds of formula (I) described hereinbefore can be shown by a number of well established tests/models, including but not limited to a GERD model in dogs.

### GERD Model in Dogs:

Beagle dogs are equipped with a chronic esophagostomy to allow passage of a manometric catheter and a pH probe along the esophagus and the stomach.

Following recording of basal pressures of the Lower Esophageal Sphincter and the stomach, one of the compound of formula (I) described hereinbefore is administered at doses of 0.03, 0.1, 0.3 and 1 mg/kg Lv. Transient Lower Esophageal Sphincter Relaxations (TLESRs) and acid reflux are induced by infusion of an acidified meal followed by stomach distention using a peristaltic pump infusing air at 40 ml/min, according to a modification of Stakeberg, J. and Lehmann, A. Neurogastroenterol. Mot. (1999) 11: 125-132. Compounds of formula (I) described hereinbefore reduce dose-dependently the frequency of TLESRs and TLESRs associated with acid reflux.

Hence, it follows that compounds of formula (I) described hereinbefore are useful in the treatment of gastro-esophageal reflux disease (GERD).

### Preparation of the Pharmaceutical Compositions:

### EXAMPLE I

Tablets of the following composition are produced in a conventional manner:

| | mg/Tablet |
|---|---|
| Active ingredient | 100 |
| Powdered. lactose | 95 |
| White corn starch | 35 |
| Polyvinylpyrrolidone | 8 |
| Na carboxymethylstarch | 10 |
| Magnesium stearate | 2 |
| Tablet weight | 250 |

### EXAMPLE II

Tablets of the following composition are produced in a conventional manner:

| | mg/Tablet |
|---|---|
| Active ingredient | 200 |
| Powdered. lactose | 100 |
| White corn starch | 64 |
| Polyvinylpyrrolidone | 12 |
| Na carboxymethylstarch | 20 |
| Magnesium stearate | 4 |
| Tablet weight | 400 |

### EXAMPLE III

Capsules of the following composition are produced:

| | mg/Capsule |
|---|---|
| Active ingredient | 50 |
| Crystalline. lactose | 60 |
| Microcrystalline cellulose | 34 |
| Talc | 5 |
| Magnesium stearate | 1 |
| Capsule fill weight | 150 |

The active ingredient having a suitable particle size, the crystalline lactose and the microcrystalline cellulose can be homogeneously mixed with one another, sieved and thereafter admixed with talc and magnesium. The final mixture is filled into hard gelatin capsules of suitable size.

1. A method of treating gastro-esophageal reflux disease (GERD) which comprises administering to an individual a therapeutically effective amount of a compound of formula (I) 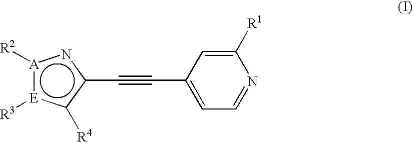
wherein
one of A or E is N and the other is C;
R¹ is halogen;
R² is C₁-C₆-alkyl;
R³ is phenyl, pyridinyl, pyrazinyl, pyrimidinyl or pyridazinyl, each of which is optionally substituted by one, two or three substituents, selected from the group consisting of halogen, C₁-C₆-alkyl, C₁-C₆-alkoxy, C₁-C₆-haloalkyl and C₁-C₆-haloalkoxy;
R⁴ is CHF₂, CF₃, CH₂OH or C₁-C₆-alkyl;
in free base or acid addition salt form.

2. The method of claim 1, wherein the compound administered has formula (Ia)

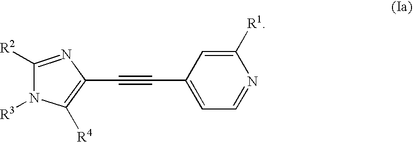

3. The method of claim 2, wherein
R¹ is halogen;
R² is methyl or i-propyl;
R³ is phenyl or pyridinyl, each of which is optionally substituted by one or more chloro, fluoro, C₁-C₆-alkyl, C₁-C₆-alkoxy, C₁-C₆-haloalkyl and C₁-C₆-haloalkoxy; and
R⁴ is methyl, CHF₂ or CH₂OH.

4. The method of claim 2, wherein R³ is unsubstituted or substituted pyridinyl, and wherein the substituents are selected from chloro, fluoro, CF₃ and C₁-C₆-alkyl.

5. The method of claim 4, wherein the compound administered is selected from the group consisting of:
2-[4-(2-Chloro-pyridin-4-ylethynyl)-2,5-dimethyl-1H-imidazol-1-yl]-5-methyl-pyridine;
2-Chloro-5-[4-(2-Chloro-pyridin-4-ylethynyl)-2,5-dimethyl-1H-imidazol-1-yl]-pyridine;
2-[4-(2-Chloro-pyridin-4-ylethynyl)-2,5-dimethyl-1H-imidazol-1-yl]-6-methyl-4-trifluoromethyl-pyridine;
2-[4-(2-Chloro-pyridin-4-ylethynyl)-2,5-dimethyl-1H-imidazol-1-yl]-6-methyl-pyridine;
2-[4-(2-Chloro-pyridin-4-ylethynyl)-2,5-dimethyl-1H-imidazol-1-yl]-6-(trifluoromethyl)-pyridine; and
3-[4-(2-Chloro-pyridin-4-ylethynyl)-2,5-dimethyl-1H-imidazol-1-yl]-5-fluoro-pyridine.

6. The method of claim 2, wherein R³ is phenyl, substituted by one or more chloro, fluoro, CF₃, C₁-C₆-alkyl, C₁-C₆-alkoxy, C₁-C₆-haloalkyl or C₁-C₆-haloalkoxy.

7. The method of claim 6, wherein the compound administered is selected from the group consisting of:
2-Chloro-4-[1-(4-fluoro-phenyl)-2,5-dimethyl-1H-imidazol-4-ylethynyl]-pyridine;
2-Chloro-4-[1-(2,4-difluoro-phenyl)-2,5-dimethyl-1H-imidazol-4-ylethynyl]-pyridine;
2-Chloro-4-[1-(3,5-difluoro-phenyl)-2,5-dimethyl-1H-imidazol-4-ylethynyl]-pyridine;
2-Chloro-4-[1-(4-fluoro-2-methyl-phenyl)-2,5-dimethyl-1H-imidazol-4-ylethynyl]-pyridine;
2-Chloro-4-[1-(4-fluoro-3-methyl-phenyl)-2,5-dimethyl-1H-imidazol-4-ylethynyl]-pyridine;
2-Chloro-4-(2,5-dimethyl-1-p-tolyl-1H-imidazol-4-ylethynyl)-pyridine;
2-Chloro-4-[1-(3-chloro-4-methyl-phenyl)-2,5-dimethyl-1H-imidazol-4-ylethynyl]-pyridine;
2-Chloro-4-[1-(3-fluoro-4-methoxy-phenyl)-2,5-dimethyl-1H-imidazol-4-ylethynyl]-pyridine;
2-Chloro-4-[1-(4-methoxy-phenyl)-2,5-dimethyl-1H-imidazol-4-ylethynyl]-pyridine;
2-Chloro-4-[2,5-dimethyl-1-(4-trifluoromethoxy-phenyl)-1H-imidazol-4-ylethynyl]-pyridine;
2-Chloro-4-[2,5-dimethyl-1-(3-trifluoromethoxy-phenyl)-1H-imidazol-4-ylethynyl]-pyridine; and
2-Chloro-4-[2,5-dimethyl-1-(4-trifluoromethyl-phenyl)-1H-imidazol-4-ylethynyl]-pyridine.

8. The method of claim 6, wherein the compound administered is selected from the group consisting of:
2-Chloro-4-[2,5-dimethyl-1-(3-methyl-4-trifluoromethoxy-phenyl)-1H-imidazol-4-ylethynyl]-pyridine;
2-Chloro-4-[1-(4-chloro-phenyl)-2,5-dimethyl-1H-imidazol-4-ylethynyl]-pyridine;
2-Chloro-4-[1-(3-chloro-2-fluoro-phenyl)-2,5-dimethyl-1H-imidazol-4-ylethynyl]-pyridine;
2-Chloro-4-[2,5-dimethyl-1-(3-trifluoromethyl-phenyl)-1H-imidazol-4-ylethynyl]-pyridine;
2-Chloro-4-[1-(3-chloro-4-fluoro-phenyl)-2,5-dimethyl-1H-imidazol-4-ylethynyl]-pyridine;
2-Chloro-4-[2,5-dimethyl-1-(2-methyl-4-trifluoromethoxy-phenyl)-1H-imidazol-4-ylethynyl]-pyridine;
2-Chloro-4-[5-difluoromethyl-1-(4-fluoro-phenyl)-2-methyl-1H-imidazol-4-ylethynyl]-pyridine;
[5-(2-Chloro-pyridin-4-ylethynyl)-3-(4-fluoro-phenyl)-2-methyl-3H-imidazol-4-yl]-methanol;
2-Chloro-4-[1-(4-methoxy-3-trifluoromethyl-phenyl)-2,5-dimethyl-1H-imidazol-4-ylethynyl]-pyridine;
2-Chloro-4-[1-(3,5-difluoro-4-methoxy-phenyl)-2,5-dimethyl-1H-imidazol-4-ylethynyl]-pyridine;
2-Chloro-4-[1-(4-methoxy-3-trifluoromethoxy-phenyl)-2,5-dimethyl-1H-imidazol-4-ylethynyl]-pyridine; and
2-Chloro-4-[1-(3-methoxy-4-trifluoromethoxy-phenyl)-2,5-dimethyl-1H-imidazol-4-ylethynyl]-pyridine.

9. The method of claim 6, wherein the compound administered is selected from the group consisting of:
2-Chloro-4-[1-(4-fluoro-2-trifluoromethoxy-phenyl)-2,5-dimethyl-1H-imidazol-4-ylethynyl]-pyridine;
2-Chloro-4-[1-(2-fluoro-4-trifluoromethoxy-phenyl)-2,5-dimethyl-1H-imidazol-4-ylethynyl]-pyridine;
2-Chloro-4-[2,5-dimethyl-1-(4-methyl-3-trifluoromethyl-phenyl)-1H-imidazol-4-ylethynyl]-pyridine;
2-Chloro-4-[2,5-dimethyl-1-(3-methyl-4-trifluoromethyl-phenyl)-1H-imidazol-4-ylethynyl]-pyridine;
2-Chloro-4-[2,5-dimethyl-1-(3-methyl-5-trifluoromethyl-phenyl)-1H-imidazol-4-ylethynyl]-pyridine;
2-Chloro-4-[1-(3-methoxy-5-trifluoromethyl-phenyl)-2,5-dimethyl-1H-imidazol-4-ylethynyl]-pyridine;
2-Chloro-4-[1-(3-methoxy-4-trifluoromethyl-phenyl)-2,5-dimethyl-1H-imidazol-4-ylethynyl]-pyridine;
2-Chloro-4-[1-(3,5-dichloro-phenyl)-2,5-dimethyl-1H-imidazol-4-ylethynyl]-pyridine;
2-Chloro-4-[1-(3-chloro-5-methyl-phenyl)-2,5-dimethyl-1H-imidazol-4-ylethynyl]-pyridine;
2-Chloro-4-[1-(3-fluoro-5-methyl-phenyl)-2,5-dimethyl-1H-imidazol-4-ylethynyl]-pyridine;
2-Chloro-4-[1-(3-chloro-5-methoxy-phenyl)-2,5-dimethyl-1H-imidazol-4-ylethynyl]-pyridine; and
2-Chloro-4-[1-(3-fluoro-5-methoxy-phenyl)-2,5-dimethyl-1H-imidazol-4-ylethynyl]-pyridine.

10. The method of claim 1, wherein the compound administered has formula (Ib)

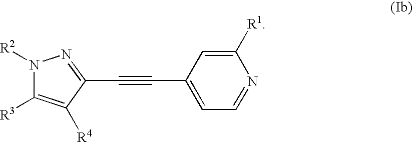

11. The method of claim 10, wherein
R¹ is halogen;
R² is methyl or i-propyl;
R³ is phenyl or pyridinyl, each of which is optionally substituted by one or more chloro, fluoro, C₁-C₆-alkyl, C₁-C₆-alkoxy, C₁-C₆-haloalkyl and C₁-C₆-haloalkoxy; and
R⁴ is methyl, CHF₂ or CH₂OH.

12. The method of claim 10, wherein wherein R³ is phenyl, substituted by one or more fluoro.

13. The method of claim 12, which is 2-Chloro-4-[5-(4-fluoro-phenyl)-1,4-dimethyl-1H-pyrazol-3-ylethynyl]-pyridine.

14. A pharmaceutical composition comprising a therapeutically effective amount of a compound of formula I 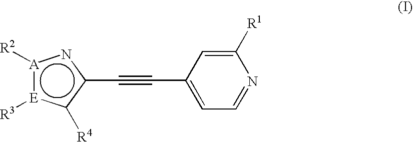
wherein
one of A or E is N and the other is C;
R¹ is halogen;
R² is C₁-C₆-alkyl;
R³ is phenyl, pyridinyl, pyrazinyl, pyrimidinyl or pyridazinyl, each of which is optionally substituted by one, two or three substituents, selected from the group consisting of halogen, C₁-C₆-alkyl, C₁-C₆-alkoxy, C₁-C₆-haloalkyl and C₁-C₆-haloalkoxy;
R⁴ is CHF₂, CF₃, CH₂OH or C₁-C₆-alkyl;
in free base or acid addition salt form and a pharmaceutically acceptable carrier.

15. The pharmaceutical composition of claim 14, further comprising at least one anti-secretory agent, selected from the group consisting of proton pump inhibitors (PPI) and histamine H2 receptor antagonists.

16. The pharmaceutical composition of claim 14, wherein the compound of formula (I) has

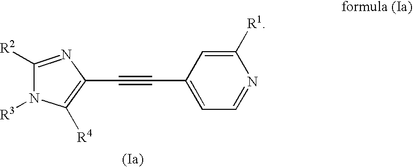

17. The pharmaceutical composition of claim 14, wherein the compound of formula (I) has formula (Ib) [chemical structure]
